(19) United States
(12) Patent Application Publication
(10) Pub. No.: US 2023/0364281 A1
(43) Pub. Date: Nov. 16, 2023

(54) CHAIR STERILIZATION DEVICE

(71) Applicant: OXTI Pte Ltd, Singapore (SG)

(72) Inventor: Chih-Feng Ho, Singapore (SG)

(21) Appl. No.: 17/744,750

(22) Filed: May 16, 2022

(51) Int. Cl. A61L 2/10; A47C 7/72; A47C 1/13

(52) U.S. Cl. CPC A61L 2/10; A47C 7/72; A47C 1/13

(57) ABSTRACT

A chair sterilization device, including: a chair, comprising a seat and seat back extending upward from the seat; and at least one sterilization lamp, configured on the chair, and including a light-emitting device, sensing device, control device and power device. With the above structure, the control device can use the sensing device to sense the usage status of the chair; when someone is away from the chair, the control device can control the light-emitting device to emit light, and when someone approaches the chair, the control device can control the light-emitting device to stop emitting light, providing the sterilization effect and preventing light from hurting the user, capable of effectively removing the germs attached to the chair.

### (a) TECHNICAL FIELD OF THE INVENTION

The present invention relates to a chair, and more particularly to a chair sterilization device provided with at least one sterilization lamp.

### (b) DESCRIPTION OF THE PRIOR ART

Bacteria and viruses are full of life; humans bring germs to every corner through contact and droplets, and environments are cleaned again to reduce the risk of germs. Frightening infectious diseases such as: tuberculosis, atypical pneumonia, Middle East Respiratory Syndrome (MERS), rabies, etc. are all transmitted by droplets and contact. Recently, people have begun to pay attention to sterilization due to the raging new crown pneumonia. People are wiping their hands and tabletops with alcohol at any time, but they will ignore the chairs that have always been in contact with them.

A conventional art, such as Taiwan Patent No. M598642, entitled “chair cooling and disinfection device”, at least includes: a chair, having a seat back and seat cushion; an electric spray can, configured on the seat back, and connected with a nozzle to spray disinfectant; an electric fan, configured on the seat back, and used to blow wind onto the seat cushion to reduce the temperature of the cushion and dry the disinfectant; a detector, configured on the chair, and used to detect whether a person sits on the cushion; and a processor, in electric connection with the detector, electric spray can and electric fan.

However, some disinfectants are corrosive, having bleaching effect, which will cause chairs to be damaged; some disinfectants have a pungent odor and are harmful to human bodies. More importantly, disinfectants are distributed nonuniformly during the spraying process, resulting in poor sterilization effect.

### SUMMARY OF THE INVENTION

To overcome the above disadvantages, the present invention proposes a chair sterilization device, including: a chair, comprising a seat and seat back extending upward from the seat; and at least one sterilization lamp, configured on the chair, and including a light-emitting device, sensing device, control device and power device,
where the sterilization lamp is installed on the seat back,
where the sterilization lamp includes a receiver, and the receiver is a wireless signal receiver,
where the sensing device is a microswitch, light sensing device or infrared sensing device,
where the sterilization lamp is a ultraviolet lamp, metal halide lamp, xenon lamp, mercury lamp or fluorescent lamp.

With the above structure, the control device can use the sensing device to sense the usage status of the chair; when someone is away from the chair, the control device can control the light-emitting device to emit light, and when someone approaches the chair, the control device can control the light-emitting device to stop emitting light, providing the sterilization effect and preventing light from hurting the user, capable of effectively removing the germs attached to the chair.

### BRIEF DESCRIPTION OF THE DRAWINGS

FIG. 1 is a perspective view of the present invention;

FIG. 2 is a perspective view of a sterilization lamp of the present invention;

FIG. 3 is a schematic view of the present invention, where the sterilization lamp stops emitting light when someone approaches a chair;

FIG. 4 is a schematic view of the present invention, where the sterilization lamp emits light when someone is away from the chair;

FIG. 5 is a block chart of the sterilization lamp of the present invention; and

FIG. 6 is a schematically perspective view of a smart phone or remote controller being used to control the sterilization lamp to emit light.

### DETAILED DESCRIPTION OF THE PREFERRED EMBODIMENTS

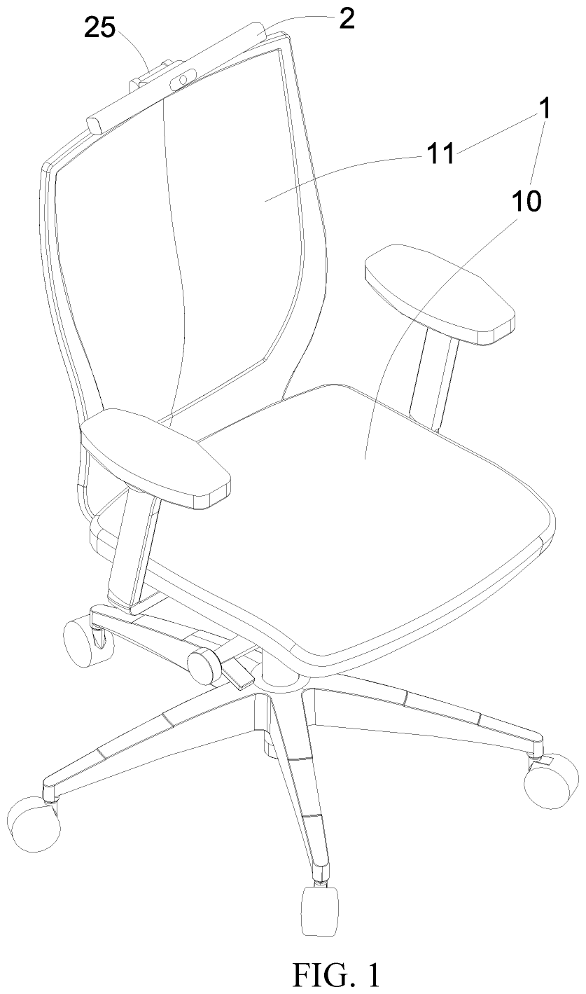

Referring to FIG. 1, a chair sterilization device includes a chair 1 and at least one sterilization lamp 2.

The chair 1 includes a seat 10 and a seat back 11 extending upward from the seat 10.

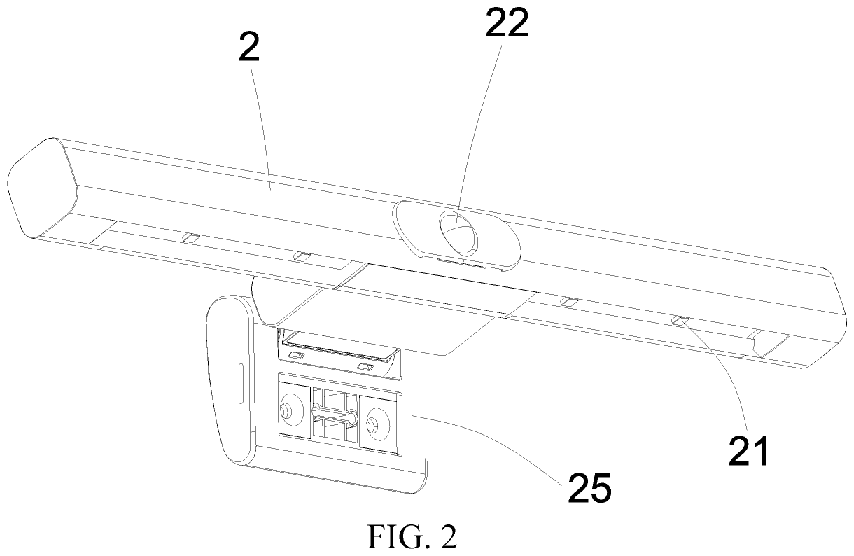

Referring to FIGS. 1, 2 and 5, the at least one sterilization lamp 2 is configured on the chair 1, and includes a light-emitting device 21, sensing device 22, control device 23 and power device 24. Specifically, the sterilization lamp 2 may be a ultraviolet lamp, metal halide lamp, xenon lamp, mercury lamp or fluorescent lamp. The sterilization lamps 2 may number one or more than one depending on requirements. In a preferred embodiment, the sterilization lamp 2 may be provided with a connection portion 25, through which the sterilization lamp 2 is connected to the chair 1, allowing the sterilization lamp 2 to be configured on the chair 1.

The light emitting device 21 may be a light bulb, light string or light emitting diode (LED) lamp used to be the light source of the sterilization lamp 2 to emit light. The sensing device 22 may be a microswitch, light sensing device or infrared sensing device used to sense the surroundings of the chair 1. The control device 23 may be a central processing unit (CPU) or microcontroller adapted to control the light emitting device 21. The power device 24 may be a battery or other power supply.

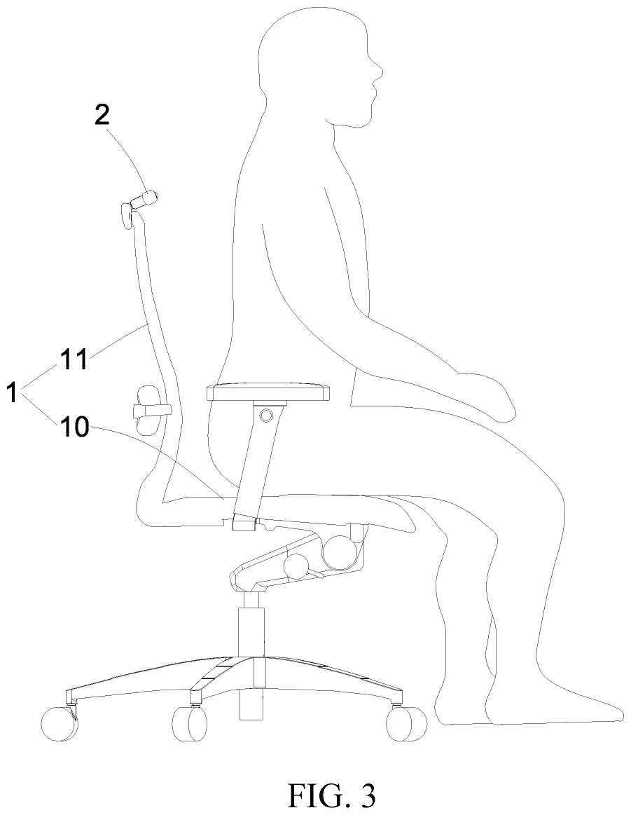

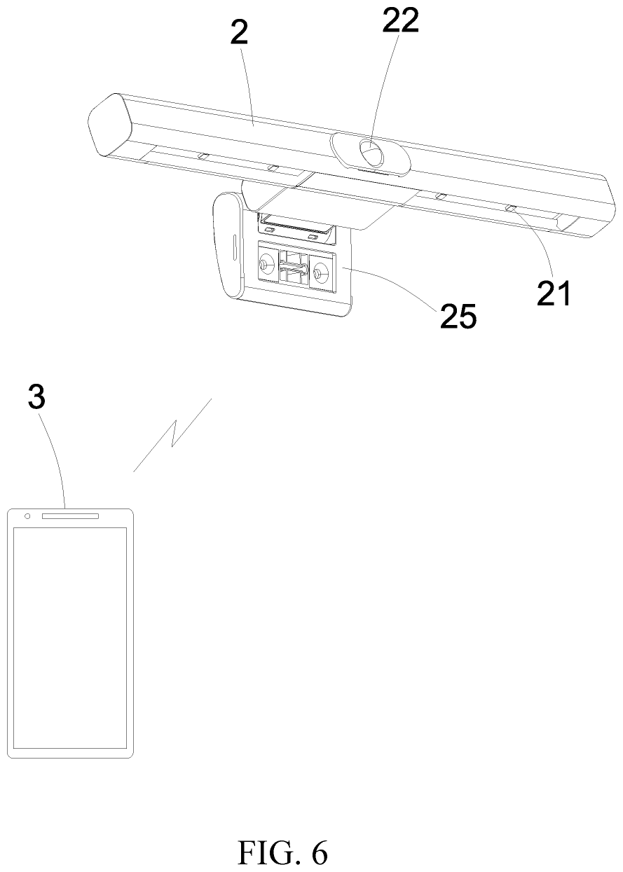

Referring to FIGS. 3, 5 and 6, in order to provide a remote-control function, the sterilization lamp 2 may further include a receiver 26, which may be a wireless signal receiver. The receiver 26 is used to receive a wireless signal, allowing the control device 23 to control the light-emitting device 21 according to an external wireless signal, which may come from a smart phone 3 or remote controller, so that the smart phone 3 or remote controller can be used to remotely control the sterilization lamp 2 to emit light.

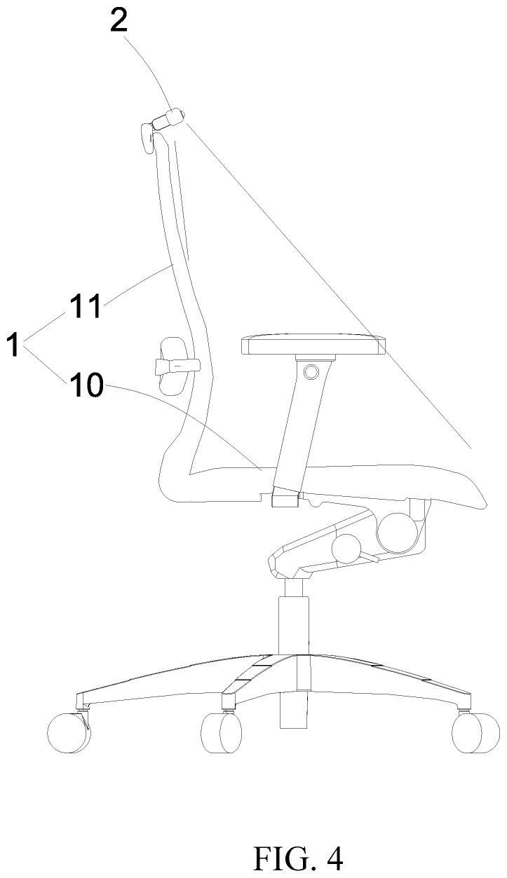

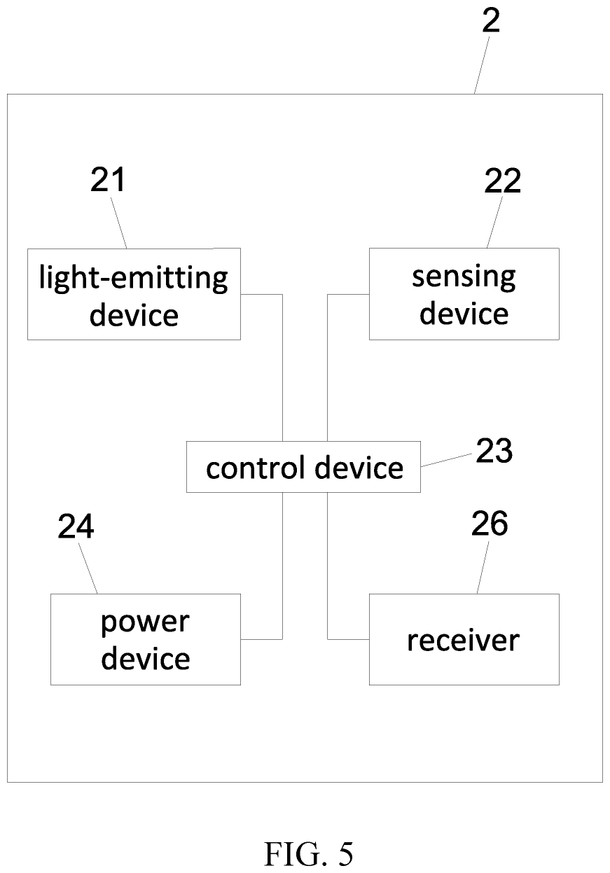

Referring to FIGS. 4 and 5, the sterilization lamp 2 is generally installed on the upper end of the seat back 11 through the connection portion 23, making the sterilization lamp 2 emit light from top to bottom, where the light emitted from the sterilization lamp 2 can cover the seat 10 and the seat back 11 to maximize the sterilization range.

In the embodiment, the sensing device 22 will detect the surroundings of the chair 1 continuously after the power supply 24 of the sterilization lamp 2 is turned on; when sensing someone approaching the chair 1, the sensing device 22 notifies the control device 23, and the control device 23 controls the light-emitting device 21 to stop emitting light, preventing the light from hurting the human body; when sensing somebody far away from the chair 1, the sensing device 22 notifies the control device 23, and the control device 23 controls the light-emitting device 21 to emit light, so that the sterilization lamp 2 can immediately emit light to illuminate the seat 10 and seat back 11 to apply sterilization.

With the above structure, the control device 23 can use the sensing device 22 to sense the usage status of the chair 1; when someone is away from the chair, the control device 23 can control the light-emitting device 21 to emit light, and when someone approaches the chair 1, the control device 23 can control the light-emitting device 21 to stop emitting light, providing the sterilization effect and preventing light from hurting the user, capable of effectively removing the germs attached to the chair 1. Furthermore, comparing to the prior arts, the light emitted from the sterilization lamp 2 of the present invention is not easy to damage the chair 1 and will not have pungent odor or corrosiveness. In addition, the light distribution is uniform inside the illumination range.

I claim:

1. A chair sterilization device, comprising:
a chair, comprising a seat and seat back extending upward from said seat; and
at least one sterilization lamp, configured on said chair, and comprising a light-emitting device, sensing device, control device and power device.

2. The chair sterilization device according to claim 1, wherein said sterilization lamp is installed on said seat back.

3. The chair sterilization device according to claim 1, wherein said sterilization lamp comprises a receiver, and said receiver is a wireless signal receiver.

4. The chair sterilization device according to claim 1, wherein said sensing device is a microswitch, light sensing device or infrared sensing device.

5. The chair sterilization device according to claim 1, wherein said sterilization lamp is a ultraviolet lamp, metal halide lamp, xenon lamp, mercury lamp or fluorescent lamp.
